(19) United States
(12) Patent Application Publication
(10) Pub. No.: US 2020/0323949 A1
(43) Pub. Date: Oct. 15, 2020

(54) MOLECULAR ENGINEERING OF A NOVEL TERNARY COMPLEX OF ACTINOMYCIN D FOR CANCER STEM CELLS TREATMENT

(71) Applicant: TRANSGENEX NANOBIOTECH, INC., Tampa, FL (US)

(72) Inventors: Mazen HANNA, Tampa, FL (US); Manomi PERERA, Tampa, FL (US); Jiyu YAN, Tampa, FL (US); Andrew HANNA, Lutz, FL (US)

(21) Appl. No.: 16/844,426

(22) Filed: Apr. 9, 2020

Related U.S. Application Data
(60) Provisional application No. 62/831,802, filed on Apr. 10, 2019.

(51) Int. Cl. A61K 38/12; A61K 9/00; A61K 31/4184; A61K 47/55; A61K 47/64

(52) U.S. Cl. CPC A61K 38/12; A61K 9/0014; A61K 47/64; A61K 31/4184; A61K 47/552; A61K 9/0019

(57) ABSTRACT

Preparation and characterization of novel DACT forms suitable for pharmaceutical compositions in drug delivery systems for humans.

### CROSS-REFERENCE TO RELATED APPLICATIONS

This application claims priority to U.S. Provisional Application No. 62/831,802, filed Apr. 10, 2019, the disclosure of which is incorporated herein by reference.

### FIELD OF THE INVENTION

This disclosure pertains to generating novel crystalline forms of Actinomycin D for the safe and effective treatment of cancers in general and cancer stem cells in particular, human diseases including a variety of cancers and particularly drug resistant cancer as well as being used as a radio sensitizer. The novel forms include but not limited to cocrystals, salts, solvates of salts, and mixtures thereof. Methods for the preparation of and pharmaceutical compositions suitable for drug delivery systems that include one or more of these new forms are also disclosed.

### BACKGROUND OF THE INVENTION

Actinomycin D also known as Dactinomycin, ActD, or DACT is one of a group of antibiotics produced by various species of Streptomyces. From all other species of Streptomyces, only Streptomyces parvullus yields an essentially pure DACT substance that contains only traces of similar compounds differing in the amino acid content of the peptide side chains. The empirical formula is C₆₂H₈₆N₁₂O₁₆, IUPAC name as 2-Amino-4,6-dimethyl-3-oxo-3H-phenoxazine-1,9-dicarboxylic acid bis-[(5,12-diisopropyl-9,13,16-trimethyl-4,7,11,14,17-pentaoxo-hexadecahydro-10-oxa-3a,6,13,16-tetraaza-cyclopentacyclohexadecen-8-yl)-amide, and the structural formula, which basically is two cyclic peptides attached to a phenoxazine.

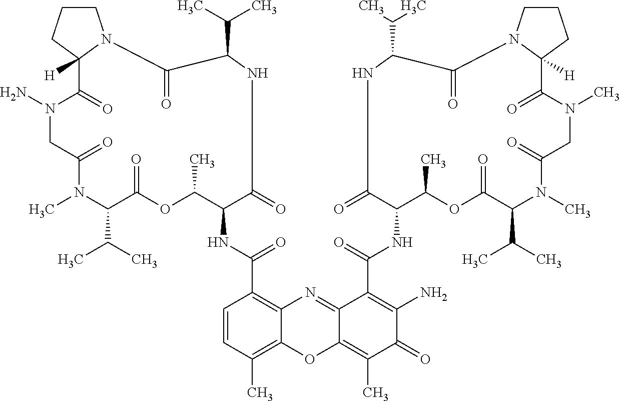 [SMILES: Cc1c2oc3c(C)ccc(C(=O)N[C@@H]4C(=O)N[C@H](C(C)C)C(=O)N5CCC[C@H]5C(=O)N(N)CC(=O)N(C)[C@@H](C(C)C)C(=O)O[C@@H]4C)c3nc-2c(C(=O)N[C@@H]2C(=O)N[C@H](C(C)C)C(=O)N3CCC[C@H]3C(=O)N(C)CC(=O)N(C)[C@@H](C(C)C)C(=O)O[C@@H]2C)c(N)c1=O]

DACT has been known for more than 70 years. It was first isolated Selman Waksman in 1940. (Waksman S A; Woodruff H B (1940) (“Bacteriostatic and bacteriocidal substances produced by soil actinomycetes”. Proc Soc Exper Biol. 45: 609-614). Additionally, it was the first antibiotic shown to have anti-cancer activity (Hollstein, U. (1974). “Actinomycin. Chemistry and mechanism of action”. Chemical Reviews. 74 (6): 625-652). More specifically, it binds to DNA and inhibits RNA synthesis (transcription), with chain elongation more sensitive than initiation, termination, or release. Due to the impaired mRNA production, protein synthesis also declines after DACT therapy. (American Medical Association; Drug Evaluations Annual, 1993, p2015).

DACT was first approved by the FDA on Dec. 10, 1964 and launched by Merck Sharp and Dohme under the trade name Cosmegen. It is supplied as a sterile, yellow, amorphous powder with 0.5 mg dose for IV use with a drug black box warning of “Highly Toxic.” It has been used to treat cancers such as gestational trophoblastic neoplasia, Wilms' tumor, rhabdomyosarcoma, Ewing's sarcoma, malignant hydatid form mole. It can also be combined with other drugs in chemotherapy regimens, like the VAC regimen with vincristine and cyclophosphamide for treating rhabdomyosarcoma and Ewing's sarcoma.

DACT is poorly absorbed from the GI tract. The drug is extremely irritating to tissues and is, therefore, administered IV. (American Society of Health System Pharmacists; AHFS Drug Information 2009. Bethesda, Md. (2009), p. 1025)

Scant literature is available on manipulation of the solid form of DACT. Crystalline forms of ethanoate/hydrate and various hydrates including an undecanoate and dodecanoate are published in the Cambridge Structural Database (CSD February 2017 Update). See Sobell et al, Nature 1971, 231, pp200, Jain et al, J Mol. Biol. 68, P1, 1972. Complexes with DNA segments are also published in CSD represented by the following DNA sequences, (ACGTAGCTACGT)2: [actinomycin D, (echinomycin)2] and d(ACGTAGCTACGT)2: [actinomycin D, (triostin A)2] (Takusagawa et al. 2000 Acta Cryst D Biol Crystalogr. 56 (3)).

No attempt has been made prior to this invention towards a deliberate molecular design to create a molecular complex of DACT (i.e., DACT and a cocrystal former) in a single crystalline structure, rather than mix the cocrystal former and DACT as a physical mix. The benefit of such design can lead to the elimination of all the batch to batch blend uniformity and particle segregation problems that powder blends often suffer from. In addition, it simplifies the manufacture of the solid dosage form that is made of a drug and excipient to a degree that the final solid dosage form is basically the powder of the molecular complex by design.

Additionally, this molecular complex will have very different physicochemical properties than that of the parent drug and co-former derived therefrom. Such properties include melting point, thermal and electrical conductivity, aqueous solubility, rate of aqueous dissolution, and potentially permeability. The new forms may also be useful to create oral dosage forms and injectables that mitigate its soft tissue toxicity. The upward trend in the use of oral drugs will continue especially in light of the goal to decrease the overall cost of healthcare. Thus, there is an opportunity to create oral dosage forms of IV drugs where oral dosage forms do not yet exist due to their poor aqueous solubility and/or poor permeability and in this case soft tissue toxicity, providing a clear clinical benefit for patients.

Furthermore, adding a second active pharmaceutical ingredient (API) to the DACT and coformer molecular complex would help further improve the potency of the DACT, reduce its dose and subsequent side effects.

### SUMMARY OF THE INVENTION

The present disclosure is directed towards generating new forms of DACT that have improved physicochemical characteristics. One aspect of the present disclosure includes novel molecular complexes of DACT neutral and ionic that includes cocrystals, salts, and solvates (e.g., hydrates and mixed solvates as well as solvates of salts), and mixtures containing such materials. In addition, the disclosure further includes methods for the preparation of such complexes.

The disclosure further includes compositions of molecular complexes of actinomycin D suitable for incorporation in a pharmaceutical dosage form. Specific molecular complexes pertaining to the disclosure include, but are not limited to, cocrystals of actinomycin D and diphenic acid, losartan, methyl and propyl parabens and telmisartan. Obvious variants of the disclosed actinomycin D forms in the text, including those described by the drawings and examples will be readily apparent to the person of ordinary skill in the art having the present disclosure, and such variants are considered to be a part of the current invention.

The disclosure also includes results of characterization of the new molecular complexes by PXRD and FTIR confirming their novelty compared with that of their parent molecule and the coformer.

The foregoing and other features and advantages of the disclosed technology will become more apparent from the following detailed description, which proceeds with reference to the accompanying drawings. Such description is meant to be illustrative, but not limiting, of the invention.

### BRIEF DESCRIPTION OF THE DRAWINGS

FIG. 1. PXRD diffractograms of actinomycin D:diphenic acid:losartan novel form.

FIG. 2. FTIR spectrum of actinomycin D:diphenic acid:losartan novel form.

FIG. 3. PXRD diffractograms of actinomycin D:diphenic acid:telmisartan novel form.

FIG. 4. FTIR spectrum of actinomycin D:diphenic acid:telmisartan novel form.

FIG. 5. PXRD diffractograms of actinomycin D:methylparaben:losartan novel form.

FIG. 6. FTIR spectrum of actinomycin D:methylparaben:losartan novel form.

FIG. 7. PXRD diffractograms of actinomycin D:methylparaben:telmisartan novel form.

FIG. 8. FTIR spectrum of actinomycin D:methylparaben:telmisartan novel form.

FIG. 9. PXRD diffractograms of actinomycin D:propylparaben:losartan novel form.

FIG. 10. FTIR spectrum of actinomycin D:propylparaben:losartan novel form.

FIG. 11. PXRD diffractograms of actinomycin D:propylparaben:telmisartan novel form.

FIG. 12. FTIR spectrum of actinomycin D:propylparaben:telmisartan novel form.

FIG. 13. PXRD diffractograms of actinomycin D:tamibarotene 1:10 molar ratio novel form.

FIG. 14 FTIR spectrum of actinomycin D:tamibarotene 1:10 molar ratio novel form.

### DETAILED DESCRIPTION OF THE INVENTION

In general, active pharmaceutical ingredients (APIs) in pharmaceutical compositions can be prepared in a variety of different forms. Such compounds can be prepared to have a variety of different chemical forms including chemical derivatives, solvates, hydrates, cocrystals, and/or salts. Such compounds can also be prepared to have different physical forms. For example, they may be amorphous, may have different crystalline polymorphs, or may exist in different solvated or hydrated states. The discovery of new forms of a pharmaceutically useful compound may provide an opportunity to improve the performance characteristics of a pharmaceutical product. Additionally, it expands the array of resources available for designing, for example, a pharmaceutical dosage form of a drug with a targeted release profile or other desired characteristics.

A specific characteristic that can be targeted includes the crystal form of an API. By altering the crystal form, it therefore becomes possible to vary the physical properties of the target molecule. For example, crystalline polymorphs typically have different aqueous solubility from one another, such that a more thermodynamically stable polymorph is less soluble than a less thermodynamically stable polymorph. In addition to water solubility, pharmaceutical polymorphs can also differ in properties such as rate of dissolution, shelf-life, bioavailability, morphology, vapor pressure, density, color, and compressibility. Accordingly, it is desirable to enhance the properties of an active pharmaceutical compound by forming molecular complexes such as a cocrystal, a salt, a solvate, or hydrate with respect to aqueous solubility, rate of dissolution, bioavailability, Cmax, Tmax, physicochemical stability, down-stream processibility (e.g., flowability compressibility, degree of brittleness, particle size manipulation), crystallization of amorphous compounds, decrease in polymorphic form diversity, toxicity, taste, production costs, and manufacturing methods.

During the development of drugs, it is frequently advantageous to have novel crystalline forms of such drug materials that possess improved properties, including increased aqueous solubility and stability. It is also desirable, in general, to increase the dissolution rate of such solid forms and potentially increase their bioavailability if used in an oral delivery setting. This also applies to the development of novel forms of DACT which, when administered orally to a subject could achieve a greater or similar bioavailability and PK profile when compared to an IV or other formulations on a dose-for-dose basis.

Cocrystals, salts, solvates, and hydrates of actinomycin D of the present invention could give rise to improved properties. For example, a new actinomycin D form for use in oral dosage forms or injectables, including direct injection and infusion, are particularly advantageous. A number of novel actinomycin D forms have been synthesized, characterized, and disclosed herein.

The present invention further includes compositions of molecular complexes of actinomycin D suitable for incorporation in a pharmaceutical dosage form. Specific molecular complexes pertaining to the disclosure include, but are not limited to, complexes of actinomycin D, diphenic acid and losartan, actinomycin D, diphenic acid and telmisartan, actinomycin D, methylparaben and losartan, actinomycin D, methylparaben and telmisartan, actinomycin D, propylparaben and losartan, actinomycin D, propylparaben, telmisartan, and actinomycin D:tamibarotene 1:10 molar ratio. Obvious variants of the disclosed actinomycin D forms in the disclosure, including those described by the drawings and examples, will be readily apparent to the person of ordinary skill in the art having the present disclosure and such variants are considered to be a part of the current invention.

In one aspect, the invention provides for a crystalline form of actinomycin D, diphenic acid and losartan. In one embodiment the actinomycin D:diphenic acid:losartan crystalline form is a solvate/hydrate.

In one embodiment, the actinomycin D:diphenic acid:losartan crystalline form is characterized by a powder X-ray diffraction peaks selected from about 4.8, 6.5, and 10.2° 2θ±0.2° 2θ. In one embodiment actinomycin D:diphenic acid:losartan crystalline form is characterized by a powder X-ray diffraction peaks comprising a powder X-ray diffraction peak at about 4.8° 2θ±0.2° 2θ. In another embodiment actinomycin D:diphenic acid:losartan crystalline form is characterized by a powder X-ray diffraction peaks comprising a powder X-ray diffraction peak at about 6.5° 2θ±0.2° 2θ. In another embodiment actinomycin D:diphenic acid:losartan crystalline form is characterized by a powder X-ray diffraction peaks disappearing a powder X-ray diffraction peak at about 10.2° 2θ±0.2° 2θ. In another embodiment actinomycin D:diphenic acid:losartan crystalline form is characterized by a powder X-ray diffraction peaks comprising any two powder X-ray diffraction peaks selected from about 4.8, 6.5, and 10.2° 2θ±0.2° 2θ. In another embodiment actinomycin D:diphenic acid:losartan crystalline form is characterized by a powder X-ray diffraction peaks comprising powder X-ray diffraction peaks selected from about 4.8, 6.5, and 10.2° 2θ±0.2° 2θ.

In another aspect, the invention provides for a crystalline form of actinomycin, diphenic acid, and telmisartan. In one embodiment, the actinomycin D:diphenic acid:telmisartan crystalline form is a cocrystal. In another embodiment, the actinomycin D:diphenic acid:telmisartan crystalline form is a solvate/hydrate. In one embodiment, the crystalline form of actinomycin D:diphenic acid:telmisartan is a 1:1:1 complex.

In another embodiment, the actinomycin D:diphenic acid:telmisartan crystalline form is characterized by a powder X-ray diffraction peaks selected from about 4.5, 6.5, 8.5, and 10.2° 2θ±0.2° 2θ. In one embodiment, actinomycin D:diphenic acid:telmisartan crystalline form is characterized by a powder X-ray diffraction peaks comprising a powder X-ray diffraction peak at about 4.5° 2θ±0.2° 2θ. In another embodiment, actinomycin D:diphenic acid:telmisartan crystalline form is characterized by a powder X-ray diffraction peaks comprising a powder X-ray diffraction peak at about 6.5° 2θ±0.2° 2θ. In another embodiment, actinomycin D:diphenic acid:telmisartan crystalline form is characterized by a powder X-ray diffraction peaks comprising a powder X-ray diffraction peak at about 8.5° 2θ±0.2° 2θ. In another embodiment, actinomycin D:diphenic acid:telmisartan crystalline form is characterized by a powder X-ray diffraction peaks disappearing a powder X-ray diffraction peak at about 10.2°2θ±0.2° 2θ. In another embodiment, actinomycin D:diphenic acid:telmisartan crystalline form is characterized by a powder X-ray diffraction peaks comprising any three powder X-ray diffraction peaks selected from about 4.5, 6.5, 8.5, and 10.2° 2θ±0.2° 2θ. In another embodiment, actinomycin D:diphenic acid:telmisartan crystalline form is characterized by a powder X-ray diffraction peaks comprising any two powder X-ray diffraction peaks selected from about 4.5, 6.5, 8.5, and 10.2° 2θ±0.2° 2θ.

In another embodiment, the actinomycin D:methylparaben:losartan crystalline form is characterized by a powder X-ray diffraction peaks selected from about 5.8, 7.8, and 10.2° 2θ±0.2° 2θ. In one embodiment, actinomycin D:methylparaben:losartan crystalline form is characterized by a powder X-ray diffraction peaks comprising a powder X-ray diffraction peak at about 5.8° 2θ±0.2° 2θ. In another embodiment, actinomycin D:methylparaben:losartan crystalline form is characterized by a powder X-ray diffraction peaks comprising a powder X-ray diffraction peak at about 7.8° 2θ±0.2° 2θ. In another embodiment, actinomycin D:methylparaben:losartan crystalline form is characterized by a powder X-ray diffraction peaks comprising a powder X-ray diffraction peak at about 10.2° 2θ±0.2° 2θ.

In another aspect, the invention provides for a crystalline form of actinomycin, methylparaben, and telmisartan. In one embodiment, the actinomycin D:methylparaben:telmisartan crystalline form is a cocrystal. In one embodiment, the actinomycin D:methylparaben:telmisartan crystalline form is a solvate/hydrate. In one embodiment, the crystalline form of actinomycin D:methylparaben:telmisartan is a 1:1:1 complex.

In another embodiment, the actinomycin D:methylparaben:telmisartan crystalline form is characterized by a powder X-ray diffraction peaks selected from about 5.8, 7.8, 11.8, and 12.2° 2θ±0.2° 2θ. In one embodiment, actinomycin D:methylparaben:telmisartan crystalline form is characterized by a powder X-ray diffraction peaks comprising a powder X-ray diffraction peak at about 5.8° 2θ±0.2° 2θ. In another embodiment, actinomycin D:methylparaben:telmisartan crystalline form is characterized by a powder X-ray diffraction peaks comprising a powder X-ray diffraction peak at about 7.8° 2θ±0.2° 2θ. In another embodiment, actinomycin D:methylparaben:telmisartan crystalline form is characterized by a powder X-ray diffraction peaks comprising a powder X-ray diffraction peak at about 11.8° 2θ±0.2° 2θ. In another embodiment, actinomycin D:methylparaben:telmisartan crystalline form is characterized by a powder X-ray diffraction peaks comprising a powder X-ray diffraction peak at about 12.3° 2θ±0.2° 2θ. In another embodiment, actinomycin D:methylparaben:telmisartan crystalline form is characterized by a powder X-ray diffraction peaks comprising any three powder X-ray diffraction peaks selected from about 5.8, 7.8, 11.8, and 12.2° 2θ±0.2° 2θ. In another embodiment, actinomycin D:methylparaben:telmisartan crystalline form is characterized by a powder X-ray diffraction peaks comprising any two powder X-ray diffraction peaks selected from about 5.8, 7.8, 11.8, and 12.2° 2θ±0.2° 2θ. In another embodiment, actinomycin D:methylparaben:telmisartan crystalline form is characterized by a powder X-ray diffraction peaks comprising powder X-ray diffraction peaks selected from about 5.8, 7.8, 11.8, and 12.2° 2θ±0.2° 2θ.

In another aspect, the invention provides for a crystalline form of actinomycin D, propylparaben, and losartan. In one embodiment, the actinomycin D:propylparaben:losartan crystalline form is a cocrystal. In one embodiment, the actinomycin D:propylparaben:losartan crystalline form is a cocrystal. In one embodiment, the actinomycin D:propylparaben:losartan crystalline form is a solvate/hydrate. In one embodiment, the crystalline form of actinomycin D:propylparaben:losartan is a 1:1:1 complex.

In another embodiment, the actinomycin D:propylparaben:losartan crystalline form is characterized by a powder X-ray diffraction peaks selected from about 5.8, 8.0, 9.5, and 15.2° 2θ±0.2° 2θ. In one embodiment, actinomycin D:propylparaben:losartan crystalline form is characterized by a powder X-ray diffraction peaks comprising a powder X-ray diffraction peak at about 5.8° 2θ±0.2° 2θ. In another embodiment, actinomycin D:propylparaben:losartan crystalline form is characterized by a powder X-ray diffraction peaks comprising a powder X-ray diffraction peak at about 8.0° 2θ±0.2° 2θ. In another embodiment, actinomycin D:propylparaben:losartan crystalline form is characterized by a powder X-ray diffraction peaks comprising a powder X-ray diffraction peak at about 9.5° 2θ±0.2° 2θ. In another embodiment, actinomycin D:propylparaben:losartan crystalline form is characterized by a powder X-ray diffraction peaks comprising a powder X-ray diffraction peak at about 15.2° 2θ±0.2° 2θ. In another embodiment, actinomycin D:propylparaben:losartan crystalline form is characterized by a powder X-ray diffraction peaks comprising any three powder X-ray diffraction peaks selected from about 5.8, 8.0, 9.0, and 15.2° 2θ±0.2° 2θ. In another embodiment, actinomycin D:propylparaben:losartan crystalline form is characterized by a powder X-ray diffraction peaks comprising any two powder X-ray diffraction peaks selected from about 5.8, 8.0, 9.0, and 15.2° 2θ±0.2° 2θ.

In another aspect, the invention provides for a crystalline form of actinomycin D, propylparaben, and telmisartan. In one embodiment, the actinomycin D:propylparaben:telmisartan crystalline form is a cocrystal. In one embodiment, the actinomycin D:propylparaben:telmisartan crystalline form is a cocrystal. In one embodiment, the actinomycin D:propylparaben:telmisartan crystalline form is a solvate/hydrate. In one embodiment, the crystalline form of actinomycin D:propylparaben:telmisartan is a 1:1:1 complex.

In another embodiment, the actinomycin D:propylparaben:telmisartan crystalline form is characterized by a powder X-ray diffraction peaks selected from about 7.8, 9.8, 11.8, and 12.5° 2θ±0.2° 2θ. In one embodiment actinomycin D:propylparaben:telmisartan crystalline form is characterized by a powder X-ray diffraction peaks comprising a powder X-ray diffraction peak at about 7.8° 2θ±0.2° 2θ. In another embodiment, actinomycin D:propylparaben:telmisartan crystalline form is characterized by a powder X-ray diffraction peaks comprising a powder X-ray diffraction peak at about 9.8° 2θ±0.2° 2θ. In another embodiment, actinomycin D:propylparaben:telmisartan crystalline form is characterized by a powder X-ray diffraction peaks comprising a powder X-ray diffraction peak at about 11.8° 2θ±0.2° 2θ. In another embodiment, actinomycin D:propylparaben:telmisartan crystalline form is characterized by a powder X-ray diffraction peaks comprising a powder X-ray diffraction peak at about 12.5° 2θ±0.2° 2θ. In another embodiment, actinomycin D:propylparaben:telmisartan crystalline form is characterized by a powder X-ray diffraction peaks comprising any three powder X-ray diffraction peaks selected from about 7.8, 9.8, 11.8, and 12.5° 2θ±0.2° 2θ. In another embodiment, actinomycin D:propylparaben:telmisartan crystalline form is characterized by a powder X-ray diffraction peaks comprising any two powder X-ray diffraction peaks selected from about 7.8, 9.8, 11.8, and 12.5° 2θ±0.2° 2θ.

In one embodiment, the crystalline form is actinomycin D:tamibarotene crystalline form. In one embodiment, the crystalline form of actinomycin D:tamibarotene is a 1:10 complex. In another embodiment, the actinomycin D:tamibarotene crystalline form is a co-crystal. In one embodiment, the actinomycin D:tamibarotene crystalline form is characterized by a powder X-ray diffraction peaks selected from about 10.8, 12.2, 16.2, 18.0, and 24.2° 2θ±0.2° 2θ. In one embodiment, actinomycin D:tamibarotene crystalline form is characterized by a powder X-ray diffraction peaks comprising a powder X-ray diffraction peak at about 10.8° 2θ±0.2° 2θ. In another embodiment, actinomycin D:tamibarotene crystalline form is characterized by a powder X-ray diffraction peaks comprising a powder X-ray diffraction peak at about 12.2° 2θ±0.2° 2θ. In another embodiment, actinomycin D:tamibarotene crystalline form is characterized by a powder X-ray diffraction peaks comprising a powder X-ray diffraction peak at about 16.2° 2θ±0.2° 2θ. In another embodiment, actinomycin D:tamibarotene crystalline form is characterized by a powder X-ray diffraction peaks comprising a powder X-ray diffraction peak at about 18.0° 2θ±0.2° 2θ. In another embodiment, actinomycin D:tamibarotene crystalline form is characterized by a powder X-ray diffraction peaks comprising a powder X-ray diffraction peak at about 24.2° 2θ±0.2° 2θ. In another embodiment, actinomycin D:tamibarotene crystalline form is characterized by a powder X-ray diffraction peaks comprising any four powder X-ray diffraction peaks selected from about 10.8, 12.2, 16.2, 18.0, and 24.2° 2θ±0.2° 2θ. In another embodiment, actinomycin D:tamibarotene crystalline form is characterized by a powder X-ray diffraction peaks comprising any three powder X-ray diffraction peaks selected from about 10.8, 12.2, 16.2, 18.0, and 24.2° 2θ±0.2° 2θ. In another embodiment, actinomycin D:tamibarotene crystalline form is characterized by a powder X-ray diffraction peaks comprising any two powder X-ray diffraction peaks selected from about 10.8, 12.2, 16.2, 18.0, and 24.2° 2θ±0.2° 2θ. In another embodiment, actinomycin D:tamibarotene crystalline form is characterized by a powder X-ray diffraction peaks comprising a powder X-ray diffraction peaks selected from about 10.8, 12.2, 16.2, 18.0, and 24.2° 2θ±0.2° 2θ.

The present invention includes complexes of actinomycin D, methylparaben, propylparaben, losartan, diphenic acid, telmisartan, and tamibarotene 1:10 molar ratio, which are capable of complexing in the solid-state, for example, through dry or solvent-drop grinding, heating or solvent evaporation of their solution in single or mixed solvent systems, slurry suspension, antisolvent, supercritical fluids, or other techniques known to a person skilled in the art. Solvents and antisolvents used to make the crystalline forms include acetone, ethanol, methanol, ethylacetate (EtOAc), isopropanol (IPA), isopropylacetate (IPAc), diethoxymethane (DEM), Toluene, BuOAc, N-methylpyrrolidone (NMP), and a heptane.

In one embodiment, the invention includes crystalline forms of actinomycin D, methylparaben, propylparaben, losartan, diphenic acid, telmisartan, and tamibarotene 1:10 molar ratio, which are capable of complexing through solvent evaporation of their solution in single or mixed solvent systems, and slurry suspension.

In another aspect, the invention provides for a pharmaceutical composition comprising molecular complexes of actinomycin D, methylparaben, propylparaben, losartan, diphenic acid, telmisartan, and tamibarotene 1:10 molar ratio. In one embodiment, the actinomycin D:diphenic acid:losartan molecular complex is a cocrystal. In another embodiment, the actinomycin D:diphenic acid:losartan molecular complex is a solvate/hydrate. In one embodiment, the actinomycin D:diphenic acid:telmisartan molecular complex is a cocrystal. In another embodiment, the actinomycin D:diphenic acid:telmisartan molecular complex is a solvate/hydrate. In one embodiment, the actinomycin D:methylparaben:losartan molecular complex is a cocrystal. In another embodiment, the actinomycin D:methylparaben:losartan molecular complex is a solvate/hydrate. In one embodiment, the actinomycin D:methylparaben:telmisartan molecular complex is a cocrystal. In another embodiment, the actinomycin D:methylparaben:telmisartan molecular complex is a solvate/hydrate. In one embodiment, the actinomycin D:propylparaben:losartan molecular complex is a cocrystal. In another embodiment, the actinomycin D:propylparaben:losartan molecular complex is a solvate/hydrate. In one embodiment, the actinomycin D:propylparaben:telmisartan molecular complex is a cocrystal. In another embodiment, the actinomycin D:propylparaben:telmisartan molecular complex is a solvate/hydrate.

In one embodiment, the actinomycin D:tamibarotene 1:10 molar ratio, molecular complex is a cocrystal. In one embodiment, the actinomycin D:tamibarotene 1:10 molar ratio, molecular complex is a solvate/hydrate.

In some embodiments, a pharmaceutical composition of the present invention is delivered to a subject via intratumoral injection. “Intratumoral injection” is a route of administration by which a pharmaceutical composition, is delivered directly to the tumor via an injection device (e.g., needle and syringe). In some embodiments, a pharmaceutical composition of the present invention is delivered to a subject via a parenteral route, an enteral route, or a topical route.

Examples of parental routes include, without limitation, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratympanic, intrauterine, intravascular, intravenous (bolus or drip), intraventricular, intravesical, and subcutaneous.

Enteral routes of administration include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories.

Topical, including transdermal, routes of administration include administration to a body surface, such as skin or mucous membranes. Delivery vehicles of the present disclosure may be administered topically (or transdermally) via a cream, foam, gel, lotion, or ointment, for example.

The pharmaceutical composition comprises a therapeutically effective amount of at least one of the novel molecular complexes of actinomycin D according to the invention and at least one pharmaceutically acceptable excipient. The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents, and sweeteners. The choice of excipient(s) will depend on factors such as the particular mode of administration and the nature of the dosage form. Solutions or suspensions used for intravenous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, or multiple dose vials made of glass or plastic.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid suspension, injectable suspension or solution, suppository, or topical or transdermal preparation or patch. The pharmaceutical formulations generally contain about 1% to about 99% by weight of at least one novel molecular complex of DACT of the invention and 99% to 1% by weight of a suitable pharmaceutical excipient. In one embodiment, the dosage form is an oral dosage form. In another embodiment, the dosage form is a parenteral dosage form. In one embodiment, the pharmaceutical dosage form is a unit dose. The term “unit dose” refers to the amount of API administered to a patient in a single dose.

The novel molecular complexes of DACT are therapeutically useful for the treatment and/or prevention of a disease for which it is indicated, e.g., cancer. Accordingly, in another aspect, the invention also relates to methods of treatment using novel molecular complexes of DACT and, or a pharmaceutical formulation containing them. As used herein, the terms “treat,” “treating,” or “treatment” means to alleviate, reduce or abrogate one or more symptoms or characteristics of a disease and may be curative, palliative, prophylactic or slow the progression of the disease. The term “therapeutically effective amount” is intended to mean that amount of drug that will elicit a desired biological or pharmacological response, i.e., an amount sufficient to treat said disease. The term “patient” includes mammals, especially humans. In one embodiment, the patient is a human. In another embodiment, the patient is a human male. In another embodiment, the patient is a human female.

In one embodiment, the invention provides for a method of treating pre-cancer or cancer comprising the step of administering to a cancer patient a therapeutically effective amount of a pharmaceutical composition of the present invention. The present invention further provides for a medicament comprising a pharmaceutical composition of the present invention for use in treating pre-cancer or cancer.

The dosage may vary depending upon the dosage form employed, sensitivity of the patient, and the route of administration. Dosage and administration are adjusted to provide sufficient levels of the active agent(s) or to maintain the desired effect. Factors, which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In some embodiments, the cancer is selected from: Wilms' tumor, rhabdomyosarcoma, lung, breast, colon, rectal head and neck, brain, pancreatic, ovarian cancer (e.g., germ cell), gestational trophoblastic neoplasm, Ewing's sarcoma, metastatic testicular tumors (e.g., nonseminoatous), gestational trophoblastic neoplasm, locally recurrent or locoregional solid tumors (sarcomas, carcinomas, and adenocarcinomas), acute myeloid leukemia (AML), multiple myeloma, prostate cancer, skin cancer, actinic keratosis, Bowen's disease, adjuvant cancer therapy, or neoadjuvant cancer therapy. In a preferred embodiment, the cancer is skin cancer, actinic keratosis, or Bowen's disease. In a further embodiment, the skin cancer is selected from the group consisting of: basal cell carcinoma (BCC), squamous cell carcinoma (SCC), and melanoma. In another embodiment, the cancer is prostate cancer. In a further embodiment, the prostate cancer is selected from the group consisting of: acinar adenocarcinoma, ductal adenocarcinoma, transitional cell (or urothelial) cancer, squamous cell cancer, small cell prostate cancer, carcinoid, and sarcoma.

### EXAMPLES

The techniques and approaches set forth in the present disclosure can further be used by the person of ordinary skill in the art to prepare variants thereof, said variants are considered to be part of the inventive disclosure.

Materials used to create the novel forms of the present inventions are commercially available and means to synthesize them as well known. DACT as a starting material used in all experiments in this disclosure was supplied by AdipoGen Life Sciences, CA, USA, with >98% purity by HPLC. All other pure chemicals (Analytical Grade) were supplied by Sigma-Aldrich and used without further purification.

Analytical techniques used to observe the crystalline forms include powder X-ray diffraction (PXRD) and Fourier transform infrared spectroscopy (FTIR). The particular methodology used in such analytical techniques herein should be viewed as illustrative, and not limiting in the context of data collection.

Powder X-Ray Diffraction (PXRD): All DACT novel molecular complex products were observed by a D-8 Bruker X-ray Powder Diffractometer using Cu Kα (λ=1.540562 Å), 40 kV, 40 mA. The data were collected over an angular range of 3° to 40° 2θ in continuous scan mode at room temperature using a step size of 0.05° 2θ and a scan speed of 6.17°/min.

FTIR analysis was performed on a Perkin Elmer Spectrum 100 FTIR spectrometer equipped with a solid-state ATR accessory.

The following examples illustrate the invention without intending to limit its scope.

### Example 1

### Preparation of Actinomycin D:Diphenic Acid:Losartan Molecular Complex

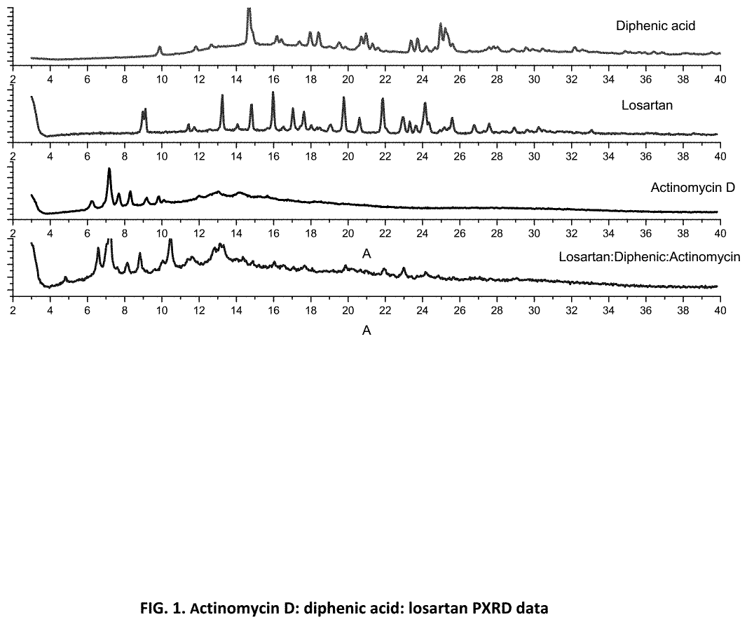

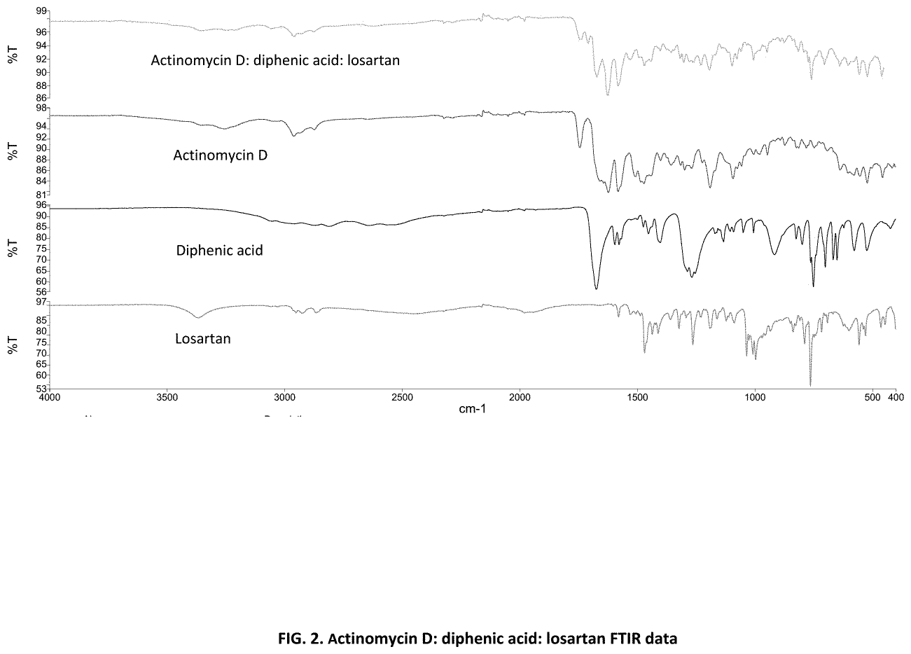

30 mg of actinomycin D, 5.8 mg of diphenic acid, and 10.1 mg of losartan (1:1:1 molar ratio) were stirred as an open slurry in 1mL of methanol in a glass vial. After 16-24 hours of stirring mixture was further dried at room temperature until a completely dried mixture is obtained. The resulted material was stored in a screw cap vial and characterized by PXRD. The materials were characterized by PXRD and FTIR corresponding to FIGS. 1 and 2, respectively.

### Example 2

### Preparation of Actinomycin D:Diphenic Acid:Telmisartan Molecular Complex

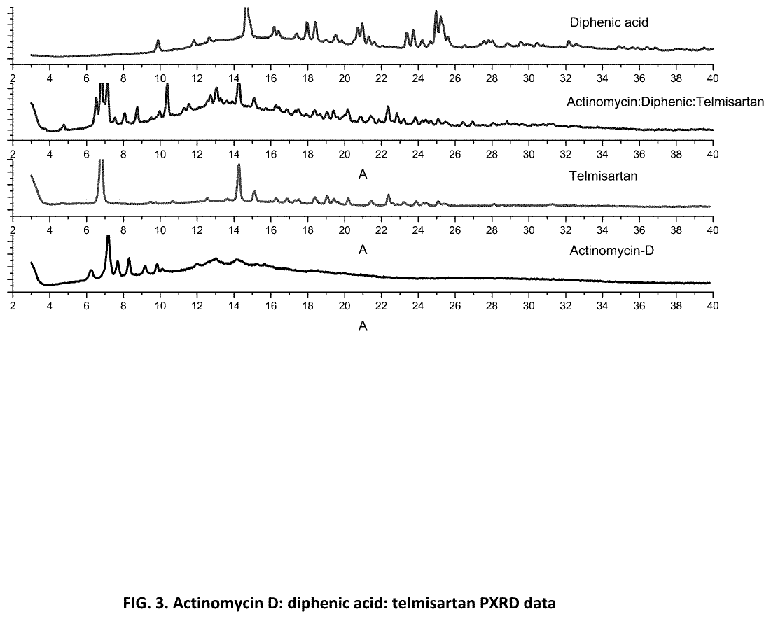

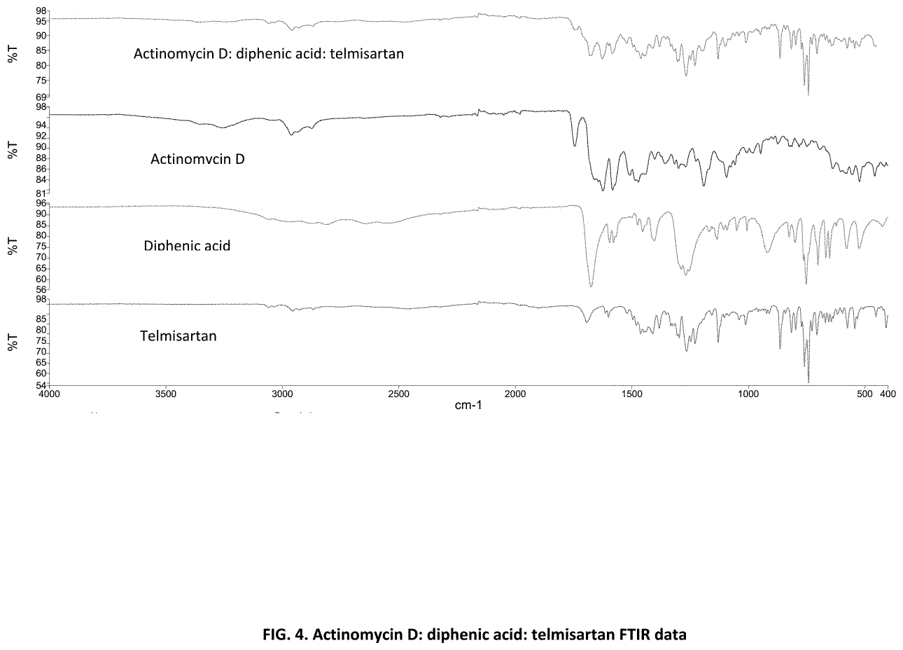

30 mg of actinomycin D, 5.8 mg of diphenic acid, and 12.3 mg of telmisartan (1:1:1 molar ratio) were stirred as an open slurry in 1 mL of methanol in a glass vial. After 16-24 hours of stirring mixture was further dried at room temperature until a completely dried mixture is obtained. The resulted material was stored in a screw cap vial and characterized by PXRD. All materials were characterized by PXRD and FTIR corresponding to FIGS. 3 and 4, respectively.

### Example 3

### Preparation of Actinomycin D:Methylparaben:Losartan Molecular Complex

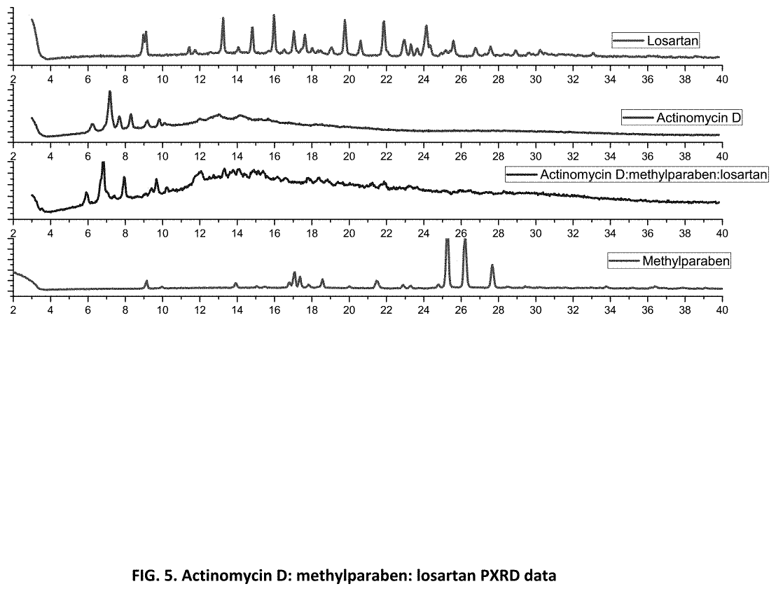

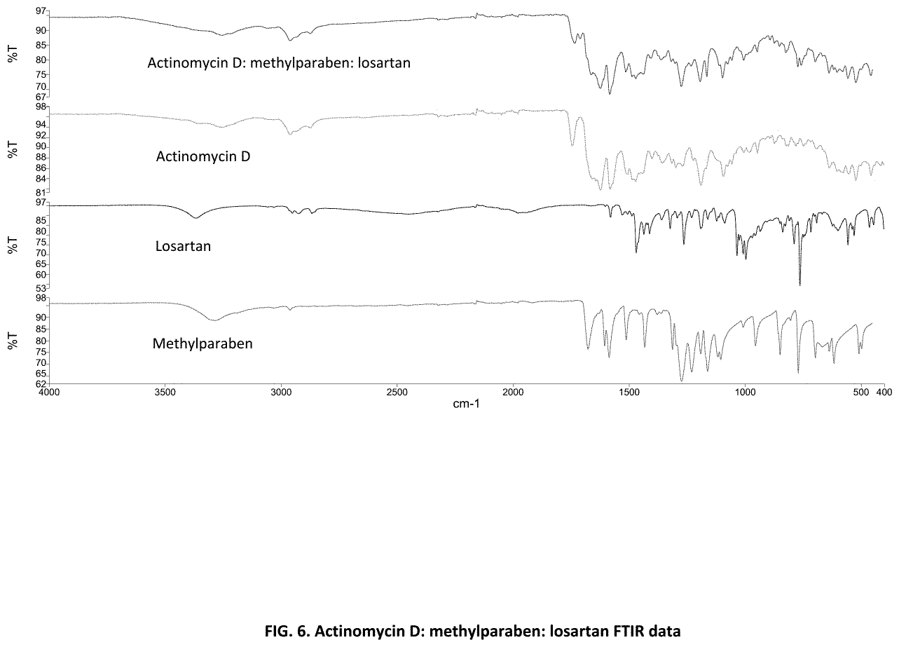

30 mg of actinomycin D, 3.6 mg of methylparaben, and 10.1 mg of losartan (1:1:1 molar ratio) were stirred as an open slurry in 1 mL of methanol in a glass vial. After 16-24 hours of stirring mixture was further dried at room temperature until a completely dried mixture is obtained. The resulted material was stored in a screw cap vial and characterized by PXRD. The material was characterized by PXRD and FTIR corresponding to FIGS. 5 and 6, respectively.

### Example 4

### Preparation of Actinomycin D:Methylparaben:Telmisartan Molecular Complex

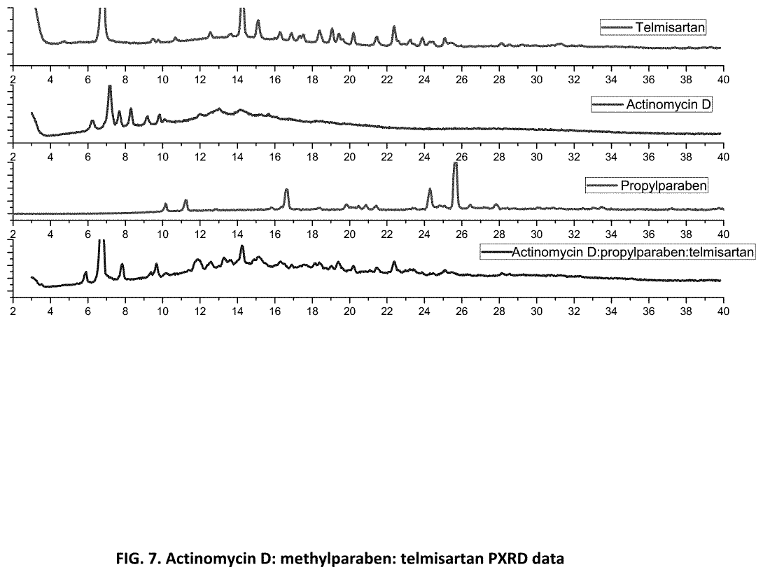

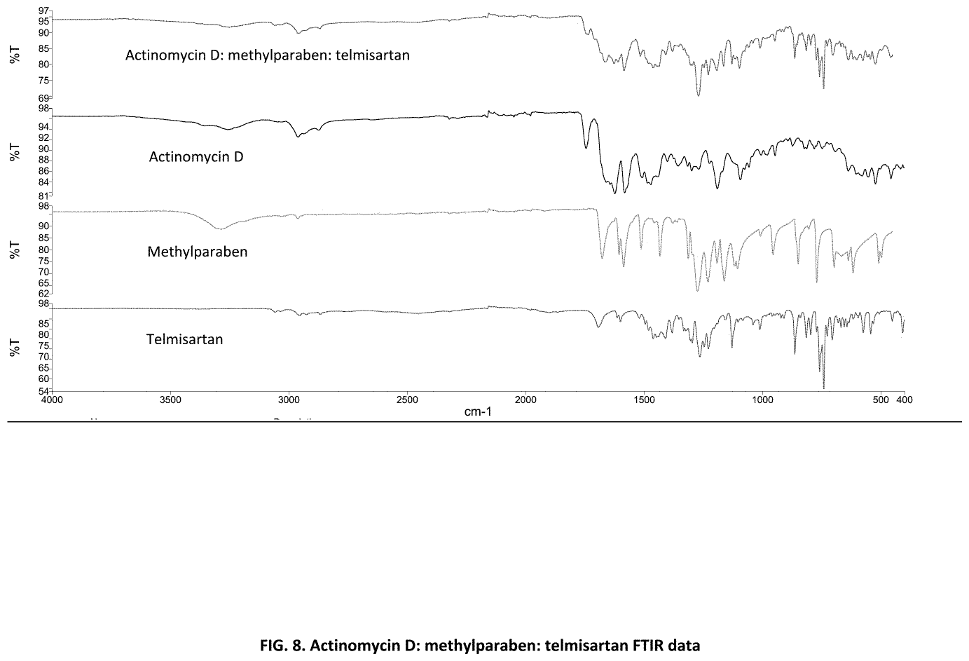

30 mg of actinomycin D, 3.6 mg of methylparaben, and 12.3 mg of telmisartan (1:1:1 molar ratio) were stirred as an open slurry in 1 mL of methanol in a glass vial. After 16-24 hours of stirring mixture was further dried at room temperature until a completely dried mixture is obtained. The resulted material was stored in a screw cap vial and characterized by PXRD. The material was characterized by PXRD and FTIR corresponding to FIGS. 7 and 8, respectively.

### Example 5

### Preparation of Actinomycin D:Propylparaben:Losartan Molecular Complex

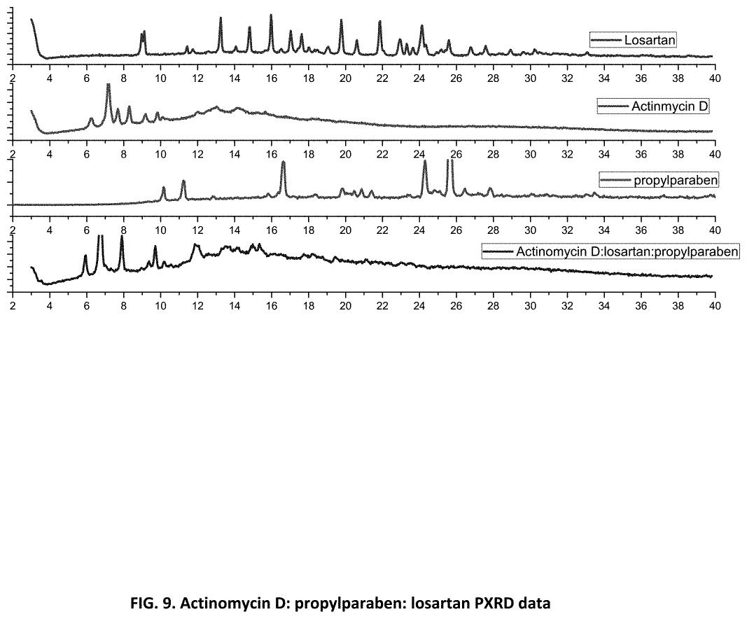

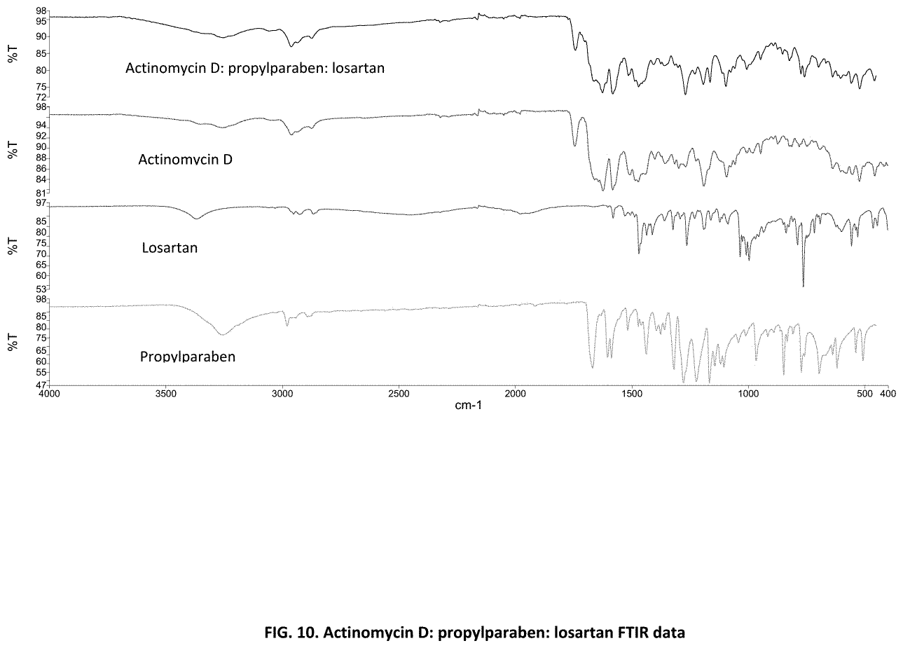

30 mg of actinomycin D, 4.3 mg of propylparaben and 10.1 mg of losartan (1:1:1 molar ratio) were stirred as an open slurry in 1 mL of methanol in a glass vial. After 16-24 hours of stirring mixture was further dried at room temperature until a completely dried mixture is obtained. The resulted material was stored in a screw cap vial and characterized by PXRD. The material was characterized by PXRD and FTIR corresponding to FIGS. 9 and 10, respectively.

### Example 6

### Preparation of Actinomycin D:Propylparaben:Telmisartan Molecular Complex

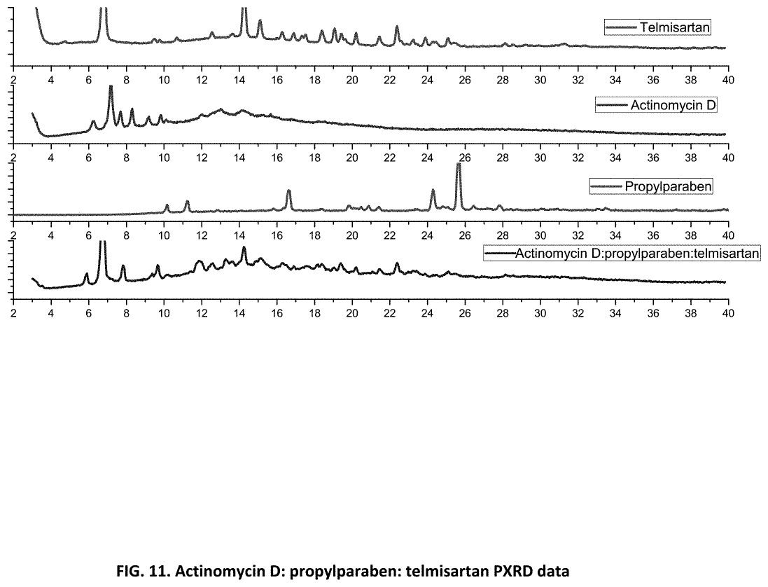

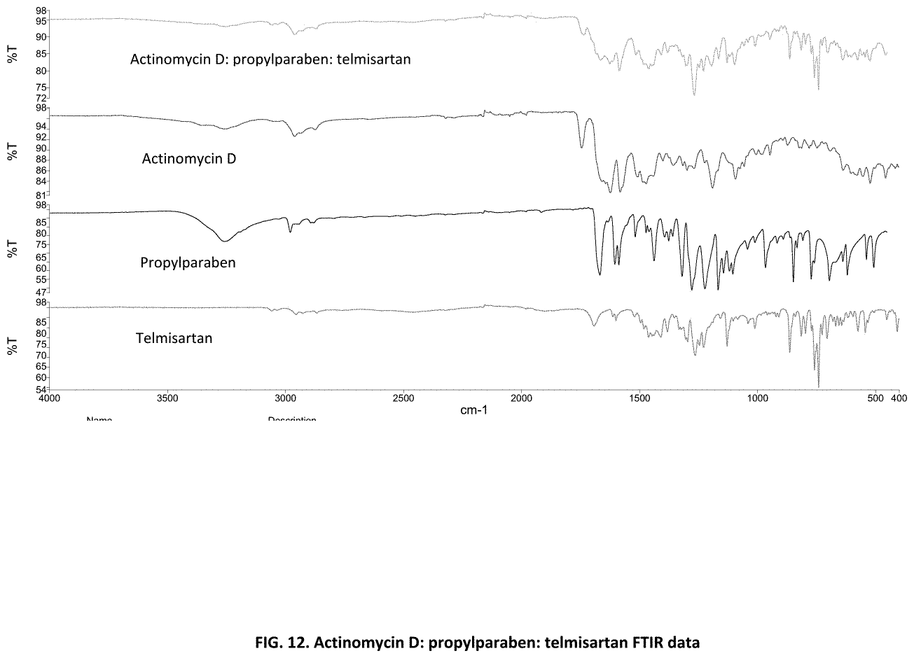

30 mg of actinomycin D, 4.3 mg of propylparaben, and 12.3 mg of telmisartan (1:1:1 molar ratio) were stirred as an open slurry in 1 mL of methanol in a glass vial. After 16-24 hours of stirring mixture was further dried at room temperature until a completely dried mixture is obtained. The resulted material was stored in a screw cap vial and characterized by PXRD. The material was characterized by PXRD and FTIR corresponding to FIGS. 11 and 12, respectively.

### Example 7

### Preparation of Actinomycin D:Tamibarotene (1:10) Complex

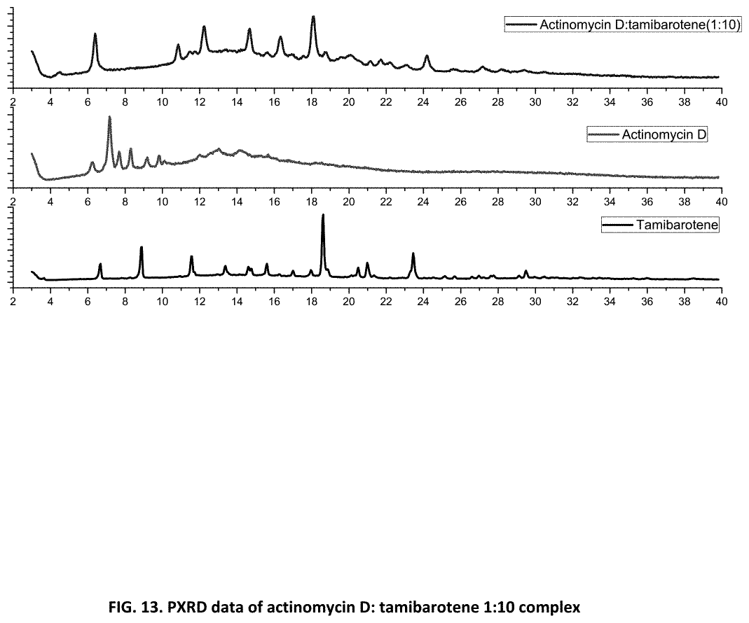

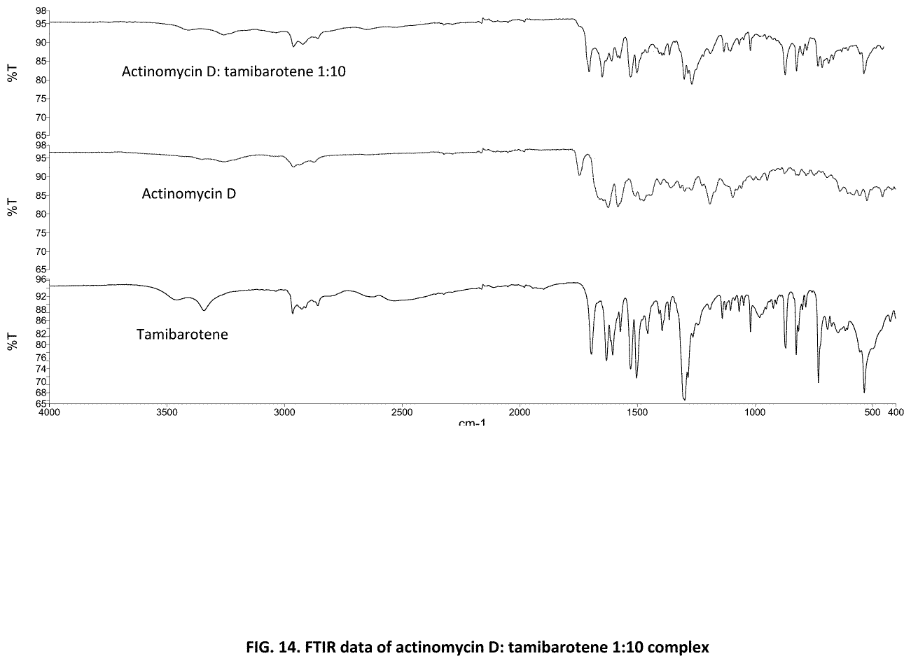

30 mg of actinomycin D and 84 mg of tamibarotene (recrystallized in acetonitrile) (1:10 molar ratio) were stirred as a slurry in an open 20 mL glass vial with 1 mL of acetone. After 12-16 hours the stirring was stopped, and the mixture was dried at room temperature for another 12-16 hours. The solids gathered, were dried and stored in a screw cap vials for subsequent analysis. The material was later characterized by PXRD and FTIR as shown in FIGS. 13 and 14, respectively.

1.-31. (canceled)

32. A crystalline form selected from the group consisting of: actinomycin D:diphenic acid:losartan, actinomycin D:diphenic acid:telmisartan, actinomycin D:methylparaben:losartan, actinomycin D:methylparaben:telmisartan, actinomycin D:propylparaben:losartan, actinomycin D:propylparaben:telmisartan, and actinomycin D:tamibarotene 1:10 molar ratio.

33. The crystalline form of claim 32, wherein said crystalline form is actinomycin D:diphenic acid:losartan.

34. The crystalline form of claim 33, wherein said crystalline form is characterized by a powder X-ray diffraction pattern comprising one or more powder X-ray diffraction peaks selected from the group consisting of about: 4.8, 6.5, and 10.2° 2θ4±0.2° 2θ.

35. The crystalline form of claim 32, wherein said crystalline form is actinomycin D:diphenic acid:telmisartan.

36. The crystalline form of claim 35, wherein said crystalline form is characterized by a powder X-ray diffraction pattern comprising one or more powder X-ray diffraction peaks selected from the group consisting of about: 4.5, 6.5, 8.5, and 10.2° 2θ±0.2° 2θ.

37. The crystalline form of claim 32, wherein said crystalline form is actinomycin D:methylparaben:losartan.

38. The crystalline form of claim 37, wherein said crystalline form is characterized by a powder X-ray diffraction pattern comprising one or more powder X-ray diffraction peaks selected from the group consisting of about: 5.8, 7.8, and 10.2° 2θ±0.2° 2θ.

39. The crystalline form of claim 32, wherein said crystalline form is actinomycin D:methylparaben:telmisartan.

40. The crystalline form of claim 39, wherein said crystalline form is characterized by a powder X-ray diffraction pattern comprising one or more powder X-ray diffraction peaks selected from the group consisting of about: 5.8, 7.8, 11.8, and 12.2° 2θ±0.2° 2θ.

41. The crystalline form of claim 32, wherein said crystalline form is actinomycin D:propylparaben:telmisartan.

42. The crystalline form of claim 41, wherein said crystalline form is characterized by a powder X-ray diffraction pattern comprising one or more powder X-ray diffraction peaks selected from the group consisting of about: 7.8, 9.8, 11.8, and 12.5° 2θ±0.2° 2θ.

43. The crystalline form of claim 32, wherein said crystalline form is actinomycin D:tamibarotene 1:10 molar ratio.

44. The crystalline form of claim 43, wherein said crystalline form is characterized by a powder X-ray diffraction pattern comprising one or more powder X-ray diffraction peaks selected from the group consisting of about: 10.8, 12.2, 16.2, 18.0, and 24.2° 2θ±0.2° 2θ.

45. A pharmaceutical composition comprising the crystalline form of claim 32 and at least one pharmaceutically acceptable excipient.

46. The pharmaceutical composition of claim 45, wherein the pharmaceutical composition is an oral dosage form, a topical dosage form, a buccal, an intranasal, an inhalable dosage form or an injectable dosage form.

47. A method of treating or preventing a disease for which actinomycin D is indicated, said method comprising the step of administering to a patient in need thereof a therapeutically effective amount of a pharmaceutical composition of claim 45.

48. The method of claim 47, wherein said disease is selected from: Wilms' tumor, rhabdomyosarcoma, lung, breast, colon, rectal head and neck, brain, pancreatic, ovarian cancer, gestational trophoblastic neoplasm, Ewing's sarcoma, metastatic testicular tumors, gestational trophoblastic neoplasm, locally recurrent or locoregional solid tumors (sarcomas, carcinomas, and adenocarcinomas), acute myeloid leukemia (AML), multiple myeloma, Shwachman-Diamond syndrome, prostate cancer, skin cancer, actinic keratosis, Bowen's disease, adjuvant cancer therapy, or neoadjuvant cancer therapy.

49. The method of claim 48, wherein said skin cancer is selected from the group consisting of: basal cell carcinoma (BCC), squamous cell carcinoma (SCC), and melanoma.

50. The method of claim 47, wherein said disease is prostate cancer and is selected from the group consisting of: acinar adenocarcinoma, ductal adenocarcinoma, transitional cell (or urothelial) cancer, squamous cell cancer, small cell prostate cancer, carcinoid, sarcoma, and Shwachman-Diamond syndrome.

51. The method of claim 47, wherein said pharmaceutical composition is administered topically or via intratumoral injection.
